(19) United States
(12) Patent Application Publication
(10) Pub. No.: US 2016/0151039 A1
(43) Pub. Date: Jun. 2, 2016

(54) ULTRASOUND DIAGNOSIS APPARATUS

(71) Applicants: Kabushiki Kaisha Toshiba, Minato-ku (JP); Toshiba Medical Systems Corporation, Otawara-shi (JP)

(72) Inventors: Koichi MORIKAWA, Nasushiobara (JP); Isao Uchiumi, Nasushiobara (JP); Nobuyuki Iwama, Nasushiobara (JP); Toru Hirano, Otawara (JP); Hironobu Hongou, Otawara (JP); Yuhei Fukuo, Nasushiobara (JP)

(73) Assignees: Kabushiki Kaisha Toshiba, Minato-ku (JP); Toshiba Medical Systems Corporation, Otawara-shi (JP)

(21) Appl. No.: 14/952,160

(22) Filed: Nov. 25, 2015

(30) Foreign Application Priority Data
Nov. 28, 2014 (JP) 2014-240791
Sep. 29, 2015 (JP) 2015-192326

(51) Int. Cl. A61B 8/08; A61B 8/06; A61B 8/00

(52) U.S. Cl. CPC A61B 8/0841; A61B 8/5207; A61B 8/461; A61B 8/06; A61B 8/523

(57) ABSTRACT

According to one embodiment, an ultrasound diagnosis apparatus includes a transmitter, a receiver, a needle tip position acquisition unit, a ROI setting unit, an image generator, and a display controller. The transmitter transmits ultrasound beams to a subject into which a puncture needle is inserted while scanning the subject. The receiver receives signals reflected from the subject. The needle tip position acquisition unit successively acquires the position of the needle tip of the puncture needle. The ROI setting unit sets a region of interest at least in a direction in which the puncture needle is inserted. The image generator generates an image of the region of interest according to the position of the needle tip based on the signals. The display controller displays the image of the region of interest.

### CROSS-REFERENCE TO RELATED APPLICATION

This application is based upon and claims the benefit of priority from Japanese Patent Application No. 2014-240791, filed Nov. 28, 2014, and No. 2015-192326, filed Sep. 29, 2015; the entire contents of which are incorporated herein by reference.

### FIELD

Embodiments described herein relate generally to an ultrasound diagnosis apparatus.

### BACKGROUND

An ultrasound diagnosis apparatus includes, for example, a transmitter, a receiver, an image generator, and a monitor. The transmitter transmits (scans) ultrasound beams in the depth direction from an ultrasound probe to a subject into which a puncture needle is inserted while sequentially switching the transmission direction in a direction perpendicular thereto (width direction). The receiver converts reflected waves received by the ultrasound probe into electrical signals and adds a time delay to the electrical signals to obtain beam signals. The image generator obtains image signals (signals indicating the intensity of the reflected waves) on a scan line extending along the depth direction based on the beam signals. The image generator stores an image signal at each point (pixel) on the scan line in a position corresponding to the position of the scan line in a frame memory as a tomographic image storage to form a tomographic image. The monitor (display) displays the tomographic image.

As used herein, the term “X direction” refers to the width direction, the term “Y direction” refers to the depth direction, and “Z direction” sometimes refers to the front-back direction perpendicular to the width direction and the depth direction. Besides, the term “B-mode image” represents a tomographic image, and the capturing of a tomographic image with an ultrasound diagnosis apparatus is sometimes referred to as “B-mode imaging”. Note that the depth direction includes the radial irradiation direction. Ultrasound beams can be irradiated radially by sequentially driving transducers as adding a delay time thereto.

Tomographic images captured by the ultrasound diagnosis apparatus are used not only for image diagnosis, but also for, for example, radiofrequency ablation (RFA) as local therapy for hepatocellular carcinoma, biopsy for examining liver cell tissue, and the like. In these therapies and examinations, a puncture needle, which is attached to an ultrasound probe, has to be inserted accurately into a region of interest such as a tumor.

For this reason, such ultrasound diagnosis apparatus is employed that allows a region of interest (ROI) having a predetermined length in two-dimensional directions, i.e., the width direction (X direction) and the depth direction (Y direction), and the puncture needle to be observed in real time. The size of the region of interest is determined such that the puncture needle is displayed on a tomographic image of the region of interest. Thereby, the operator can figure out the position in a subject up to which the puncture needle has been inserted. The puncture performed with viewing the puncture needle is sometimes referred to as “puncture under ultrasound (echo) guidance”.

In the puncture under ultrasound echo-guidance, if the tip of the puncture needle is deviated from a tomographic image of the region of interest, the needle tip is not displayed on the tomographic image. At this time, if the operator mistakenly perceives an end of the puncture needle displayed on the tomographic image as the needle tip, he/she may insert the puncture needle too far. In this case, if a blood vessel runs ahead of the needle tip, the needle tip may damage the blood vessel.

To prevent the needle tip from damaging a blood vessel, the ultrasound diagnosis apparatus operates as follows: upon receipt of beam signals from the receiver, the image generator performs quadrature phase detection of the beam signals to thereby detect only a Doppler shift frequency of the frequencies of ultrasound beams; obtains a blood-flow signal (signal indicating blood-flow velocity, distribution, and power) on a scan line based on the detected wave; and stores the blood-flow signal at each point (pixel) on the scan line in a position corresponding to the position of the scan line in the frame memory as a blood-flow image storage to form a blood-flow image. Incidentally, the blood-flow image is sometimes referred to as “Doppler image”, and the capturing of the Doppler image with the ultrasound diagnosis apparatus is sometimes referred to as “Doppler mode imaging”. In addition, the insertion route of the puncture needle expected when the puncture needle is inserted into the subject based on a direction in which the needle tip is directed or expected based on a direction in which the needle tip has been moved is referred to as “puncture guideline”. That is, the puncture guideline is successively changed based on these directions

The size of the region of interest in a blood-flow image is set such that the entire puncture guideline is accommodated therein. There has been proposed a technology (proposed technology) to display images in the number of images generated per unit time (frame rate or volume rate, hereinafter referred to as “frame rate”) by superimposing a blood-flow image captured by Doppler mode imaging on a tomographic image captured by B-mode imaging. With this, regardless of the position of the needle chip on the puncture guideline, it is possible to figure out whether a blood vessel runs ahead of the direction in which the needle chip is being directed.

However, in the above proposed technology, ultrasound beams are sequentially transmitted and received for each of scan lines, and therefore, a certain time is required to obtain beam signals necessary to generate the blood-flow image. For example, when a predetermined number of ultrasound beams are transmitted and received in the width direction to capture a blood-flow image of the region of interest, the required time increases as the length (size) of the region of interest increases in the depth direction and/or the width direction. Accordingly, the generation rate that is a reciprocal of the required time decreases. In other words, the larger the region of interest is in the blood-flow image, the lower the upper limit of the realizable generation rate becomes. If generated at a lowered upper value (low frame rate), the blood-flow image may not be displayed in real time.

To prevent the frame rate from decreasing, the operator may manually reset the position of the region of interest in the blood-flow image according to the position of the needle tip after initiating the insertion of the puncture needle. However, this requires the interruption of the puncture, which leads to a prolonged surgery, resulting in a burden on the operator.

### BRIEF DESCRIPTION OF THE DRAWINGS

FIG. 1 is a block diagram illustrating the configuration of an ultrasound diagnosis apparatus according to a first embodiment;

FIG. 2 is a schematic diagram illustrating a positional relationship among a region of interest in an ultrasound image, a region of interest in a blood flow image, and a puncture needle in the first embodiment;

FIG. 3 is an example of a rate relation table;

FIG. 4 is an example of a movement amount relation table;

FIG. 5A is an example of a three-dimensional ultrasound image of a cross-section A in the first embodiment;

FIG. 5B is an example of a three-dimensional ultrasound image of a cross-section B in the first embodiment;

FIG. 5C is an example of a three-dimensional ultrasound image of a cross-section C in the first embodiment;

FIG. 5D is a schematic diagram illustrating a positional relationship among the cross-sections A, B and C in the first embodiment;

FIG. 6 is a schematic diagram illustrating a positional relationship among a region of interest in a blood flow image, and the cross-sections A, B and C;

FIG. 7 is a timing chart illustrating the operation of each unit from the movement of the puncture needle until the display of a cross-section image;

FIG. 8 is a timing chart illustrating the operation of each unit of an ultrasound diagnosis apparatus according to a second embodiment from the movement of a puncture needle until the display of a cross-section image;

FIG. 9A is an example of a three-dimensional ultrasound image of the cross-section A in the second embodiment;

FIG. 9B is an example of a three-dimensional ultrasound image of the cross-section B in the second embodiment;

FIG. 9C is an example of a three-dimensional ultrasound image of the cross-section C in the second embodiment;

FIG. 9D is a schematic diagram illustrating a positional relationship among the cross-sections A, B and C in the second embodiment;

FIG. 10 is a block diagram illustrating the configuration of an ultrasound diagnosis apparatus according to a third embodiment; and

FIG. 11 is a timing chart illustrating the operation of each unit of an ultrasound diagnosis apparatus of the third embodiment from the movement of a puncture needle until the display of a cross-section image.

### DETAILED DESCRIPTION

In general, according to one embodiment, an ultrasound diagnosis apparatus includes a transmitter, a receiver, a needle tip position acquisition unit, a ROI setting unit, an image generator, and a display controller. The transmitter transmits ultrasound beams to a subject into which a puncture needle is inserted while scanning the subject. The receiver receives signals reflected from the subject. The needle tip position acquisition unit successively acquires the position of the needle tip of the puncture needle. The ROI setting unit sets a region of interest at least in a direction in which the puncture needle is inserted. The image generator generates an image of the region of interest according to the position of the needle tip based on the signals. The display controller displays the image of the region of interest.

In the proposed technology described above, in the puncture under ultrasound echo-guidance, to figure out whether a blood vessel runs ahead of the direction in which the needle chip is being directed regardless of the position of the needle chip on a puncture guideline, the size of a region of interest (ROI) in a blood-flow image is set such that the entire puncture guideline is accommodated therein. This necessitates an increase in the size of the region of interest. As a result, a high frame rate cannot be maintained.

On the other hand, according to the embodiment, the position of a region of interest in a blood-flow image is set according to the position of the needle tip. Thus, the region of interest in the blood-flow image can be set to a minimum necessary size that is limited to the position of the needle tip and a neighboring region thereof.

With the configuration described above, since the region of interest is set to a minimum necessary size, blood-flow images can be generated at a high frame rate. Thereby, the blood-flow images are displayed in real time. In addition, the region of interest is positioned automatically according to the position of the needle tip. This removes factors that prolong a surgery, thus imposing no burden on the operator. Further, the position of the region of interest corresponds to the position of the needle tip. Therefore, even if the needle tip is not displayed on an ultrasound image, the position of the needle tip can be determined based on the position of the region of interest. Thereby, a positional relationship can be discerned between the needle tip and a blood vessel in the region of interest. As a result, for example, it can be figured out whether a blood vessel runs ahead of the direction in which the needle chip is being directed.

Incidentally, the ultrasound image may be a two-dimensional image in the X and Y directions or a three-dimensional image in the X, Y and X directions. Besides, a blood-flow image displayed as being superimposed on the ultrasound image may also be a two-dimensional image or a three-dimensional image.

In the following, an example is described in which a three-dimensional blood-flow image is superimposed on a three-dimensional ultrasound image to be displayed.

### First Embodiment

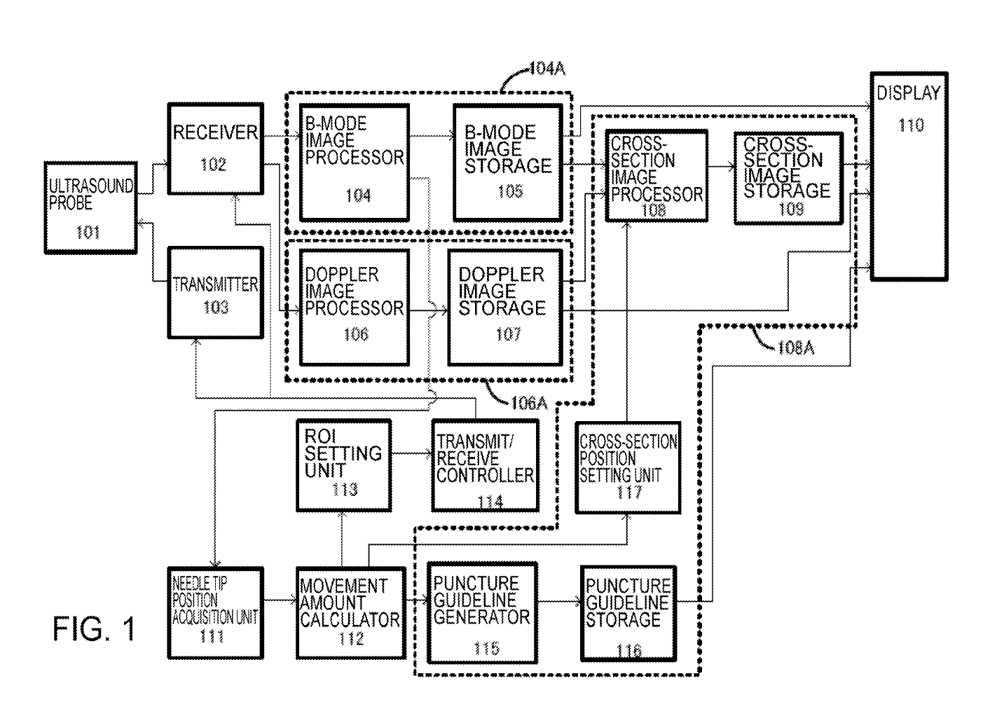

With reference to FIG. 1, a description is given of an ultrasound diagnosis apparatus according to a first embodiment. FIG. 1 is a block diagram illustrating the configuration of the ultrasound diagnosis apparatus of the first embodiment. As illustrated in FIG. 1, the ultrasound diagnosis apparatus includes an ultrasound probe 101, a receiver 102, a transmitter 103, an ultrasound image generator 104A, a blood-flow image generator 106A, a cross-section image generator 108A, a display 110, a needle tip position acquisition unit 111, a movement amount calculator 112, a ROI setting unit 113, and a transmit/receive controller 114.

The ultrasound image generator 104A includes a B-mode image processor 104 and a B-mode image storage 105. The blood-flow image generator 106A generates a blood-flow image that represents blood-flow information of a region of interest as a ROI image. The blood-flow image generator 106A includes a Doppler image processor 106 and a Doppler image storage 107. The cross-section image generator 108A includes a cross-section image processor 108, a cross-section image storage 109, a puncture guideline generator 115, a puncture guideline storage 116, and a cross-section position setting unit 117.

### (Ultrasound Probe 101, Receiver 102, Transmitter 103)

The ultrasound Probe 101 includes two-dimensional arrays of transducers that perform the conversion (electro-acoustic conversion) between electrical signals and ultrasound acoustic signals. The transmitter 103 transmits ultrasound beams in the depth direction from the transducers to a subject, into which a puncture needle NL is inserted, while sequentially switching the transmission direction to a direction (width direction) perpendicular thereto (width direction scanning). In addition, the transmitter 103 performs the width direction scanning in the front-back direction (Z direction) (front-back direction scanning). Having reached a reflection source in the subject, the ultrasound beams are reflected and return to the transducers.

The receiver 102 converts the reflected waves received by the transducers into electrical signals and adds a time delay to the electrical signals to obtain beam signals corresponding to a scan line extending along the depth direction.

### (Transmit/Receive Controller 114)

The transmit/receive controller 114 controls the transmitter 103 and the receiver 102 to perform B-mode imaging and Doppler mode imaging in time division. For example, B-mode imaging is performed once each time Doppler mode imaging is performed a predetermined number of times (e.g., four times) under the control of the transmit/receive controller 114.

In the B-mode imaging, the transmit/receive controller 114 receives a region of interest ROI2 in an ultrasound image and a frame rate R2, and obtains transmission conditions (delay time, the number of transmissions, etc.) and reception conditions (reception channel, delay amount for each channel, etc.). The transmit/receive controller 114 controls the transmitter 103 and the receiver 102 based on these conditions to repeat scan for one still image having a necessary number of scan lines for rendering a three-dimensional ultrasound image of the region of interest ROI2 at the frame rate R2.

In the Doppler mode imaging, the transmit/receive controller 114 receives regions of interest ROI1 and ROI1′ in a blood-flow image, and a frame rate R1, and obtains transmission conditions (delay time, the number of transmissions, etc.) and reception conditions (reception channel, delay amount for each channel, etc.). The transmit/receive controller 114 controls the transmitter 103 and the receiver 102 based on these conditions to transmit ultrasound beams and receive reflected waves a plurality of times (e.g., four times) per one scan line to thereby repeat scan for one still image having a necessary number of scan lines for rendering a three-dimensional blood-flow image of the regions of interest ROI1 and ROI1′ at the frame rate R1. The blood-flow image corresponds to an example of ROI image in the claims.

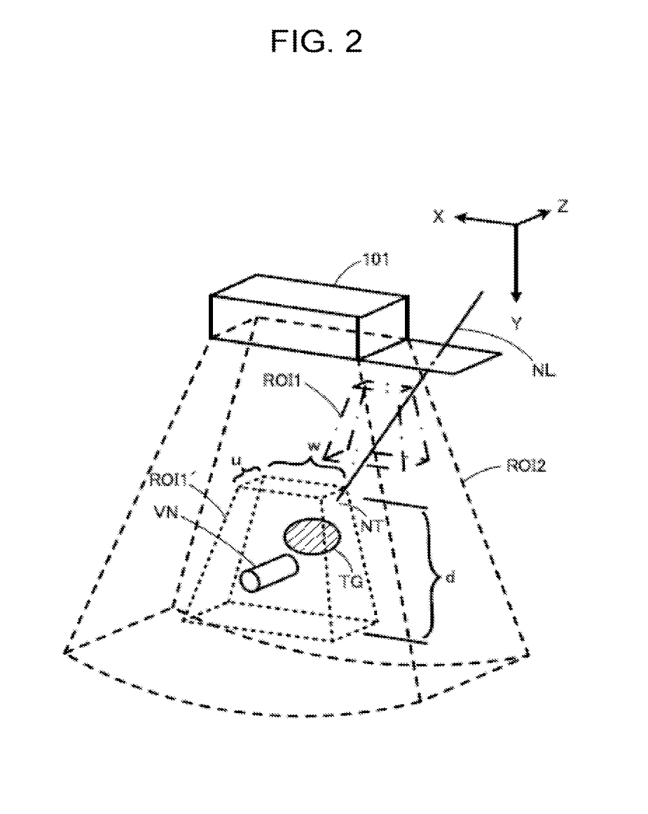

FIG. 2 is a schematic diagram illustrating a positional relationship among the regions of interest ROI1 and ROI1′ in an ultrasound image, the region of interest ROI2 in an ultrasound image, and the puncture needle NL. As described later, the position of the tip of the puncture needle is obtained per unit time (e.g., 0.05 sec.), and the region of interest is sequentially displayed in a position corresponding to the position of the needle tip. Accordingly, in common puncture, the regions of interest are sequentially displayed as overlapping one another. If they are represented in an image, the regions of interest are not clearly viewed. Therefore, in FIG. 2, the regions of interest ROI1 and ROI1′ sequentially displayed in a position corresponding to the position of the needle tip are displayed in positions apart from each other. The ultrasound probe 101 is provided with a guide mechanism having an insertion hole that guides the puncture needle NL inserted therein.

### (Needle Tip Position Acquisition Unit 111)

The needle tip position acquisition unit 111 successively acquires the position of the tip of the puncture needle NL. The needle tip position acquisition unit 111 is arranged in the guide mechanism. The needle tip position acquisition unit 111 includes a movement amount sensor (including a roller that rotates according to the movement of the puncture needle and an encoder that detects the rotation amount of the roller) and a passing sensor. The movement amount sensor detects the movement amount of the puncture needle NL when it moves along the puncture guideline. The passing sensor detects that the needle tip NT passes through a reference point on the puncture guideline. The needle tip position acquisition unit 111 is configured to obtain an insertion amount based on the movement amount from the reference point, and thereby acquire the position of the needle tip on the coordinates based on the insertion amount from the reference point on the puncture guideline. Note that the needle tip position acquisition unit 111 may acquire the position of the needle tip in any other manners. For example, the needle tip position acquisition unit 111 may be configured to acquire the position of the needle tip per unit time based on an ultrasound image where the puncture needle NL is rendered with reference to the brightness value of the end of the puncture needle NL and the shape thereof. The needle tip position acquisition unit 111 converts the coordinates of the position of the needle tip thus acquired into the coordinates of a three-dimensional ultrasound image (an image generated by the B-mode image processor 104 described later).

### (Movement Amount Calculator 112)

The movement amount calculator 112 calculates movement amount L1=P2−P1, L2=P3−P2, L3=P4−P3, . . . of the position of the needle tip NT per unit time based on the position P1, P2, P3, P4, . . . of the needle tip NT on the three-dimensional coordinates obtained per unit time by coordinate transformation. The movement amount calculator 112 also calculates movement velocity V1=(L2−L1), V2=(L3−L2), V3=(L4−L3), . . . per unit time, and direction D1=(P2−P1), D2=(P3−P2), D3=(P4−P3), . . . , in which the needle tip has been moved. The movement amount L and the movement velocity V obtained per unit time corresponds to the velocity and acceleration of the needle tip NT.

The movement amount L and the movement velocity V of the position of the needle tip NT per unit time can be obtained based on the movement amount L of the puncture needle NL that passes through the reference point detected per unit time by the movement amount sensor (described above) of the needle tip position acquisition unit 111. The movement amount L and the movement velocity V of the position of the needle tip NT per unit time can also be obtained based on the rotation angle (rotation amount) of the roller measured per unit time and an increase or a decrease in the rotation angle (rotation amount).

The size of the regions of interest ROI1 and ROI1′ in the blood-flow image is set according to the movement amount L of the position of the needle tip per unit time and the like (described in detail later). For this reason, the unit time for calculating the movement amount L and the like needs to be equal to or less than the required time for generating the blood-flow image (a reciprocal of the frame rate R1 of the blood-flow image). The upper limit of the frame rate R1 is 20 fps (described later). In this case, the unit time for detecting the movement amount L and the like is 0.05 (=1/20) second.

### (ROI Setting Unit 113)

Described below is the setting of the region of interest ROI2 and the frame rate R2 related to the ultrasound image. The ROI setting unit 113 sets at least a region of interest in the insertion direction of the needle tip. In response to an input provided through an operation unit (not illustrated) by the user before the puncture under ultrasound echo-guidance, the ROI setting unit 113 sets the region of interest ROI2 for a three-dimensional ultrasound image as well as the frame rate R2 to the transmitter 103.

During the puncture, the ROI setting unit 113 outputs the region of interest ROI2 and the frame rate R2 for the ultrasound image to the transmit/receive controller 114. Incidentally, the position and the size of the region of interest ROI2 and the frame rate R2 are constant, and do not vary depending on the movement amount L (corresponding to the velocity) of the position of the needle tip per unit time and/or the movement velocity V (corresponding to the acceleration) of the position of the needle tip per unit time. In response to an input provided through the operation unit (not illustrated) by the user, the ROI setting unit 113 changes the frame rate R2.

Described below is the setting of the regions of interest ROI1 and ROI1′ and the frame rate R1 related to the blood-flow image.

The ROI setting unit 113 outputs the initial value (depth index d3 described later) of the size of the regions of interest ROI1 and ROI1′, and the initial value (e.g., 14 fps described later) of the frame rate R1 corresponding thereto to the transmit/receive controller 114.

As illustrated in FIG. 2, assuming that the size S of the regions of interest ROI1 and ROI1′ in the blood-flow image is the product of the lengths d, w and u in the depth, width and front-back directions, the size S is represented by following expression (1):

S=d*w*u (1)

It is assumed herein that, in the regions of interest ROI1 and ROI1′ in the blood-flow image, the lengths d, w and u are increased or decreased in the same ratio. The length d in the depth direction may be set to a length according to the movement amount L and the like of the position of the needle tip per unit time, while the lengths w and u may be constant and not vary according to the movement amount L and the like.

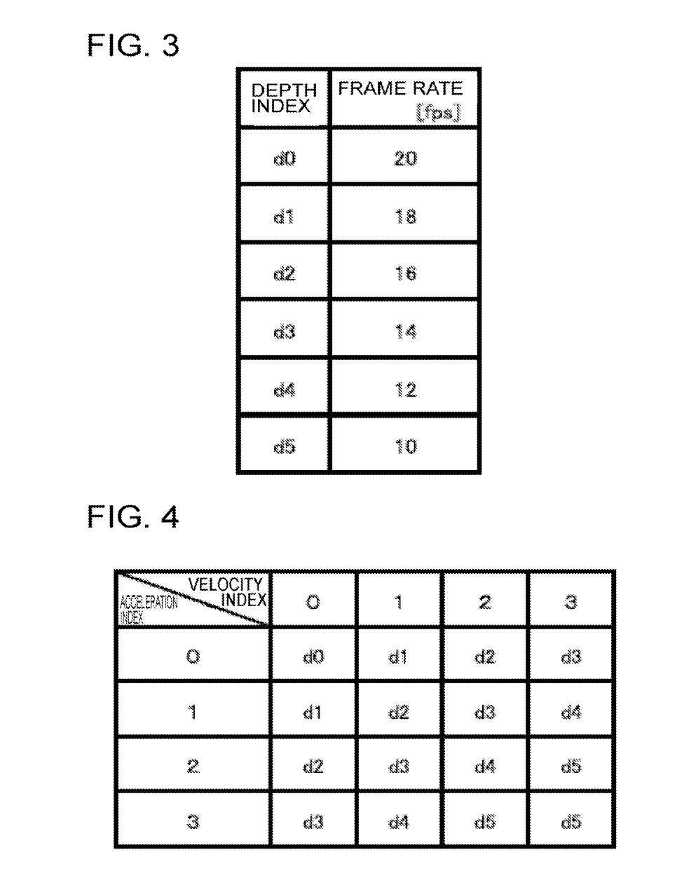

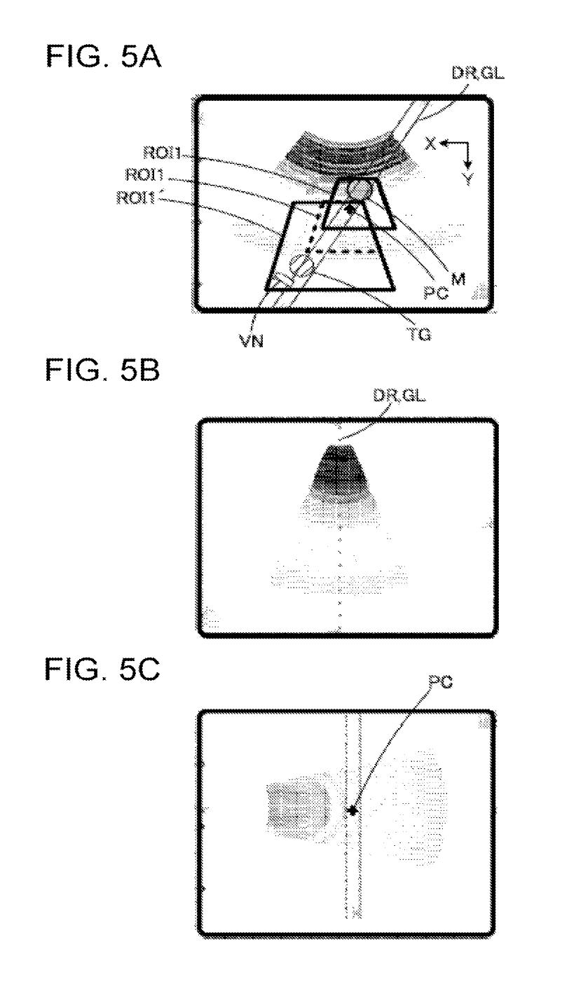

The ROI setting unit 113 stores a movement amount relation table (see FIG. 4) illustrating a corresponding relationship between velocity indices and/or acceleration indices and depth indices d0 to d5, and a rate relation table (see FIG. 3) illustrating a corresponding relationship between the depth indices and the frame rate R1. During the puncture, the ROI setting unit 113 obtains the position of the regions of interest ROI1 and ROI1′ in the blood-flow image that contains the position of the needle tip per unit time for which the needle tip position acquisition unit 111 acquires the position of the needle tip. The ROI setting unit 113 obtains the depth index as the size of the regions of interest ROI1 and ROI1′ based on the movement amount L and the movement velocity V of the needle tip NT with reference to the movement amount relation table and the rate relation table. The ROI setting unit 113 obtains the frame rate R1 from the depth index and sends the resultant to the transmit/receive controller 114. Thereby, the regions of interest ROI1 and ROI1′ are set to include the position of the needle tip, i.e., such that the upper edge of the regions of interest ROI1 and ROI1′ (the head portion of the region of interest in the blood-flow image having a substantially truncated pyramid shape) corresponds to the position of the needle tip (see FIG. 2).

FIG. 3 illustrates an example of the rate relation table. The rate relation table provides six levels of the depth indices d0 to d5 each corresponding to the value of the depth direction length d (see FIG. 2) of the regions of interest ROI1 and ROI1′ in the blood-flow image. The ROI setting unit 113 stores, as the rate relation table, values of the frame rate R1 corresponding to the depth indices d0 to d5 as the size of the regions of interest ROI1 and ROI1′ in the blood-flow image.

In the rate relation table illustrated in FIG. 3, as the depth index gradually increases from d0 to d5, the frame rate R1 [fps] gradually decreases from 20 to 10. To display the blood-flow image in real time, the frame rate R1 needs to be kept high. Accordingly, the frame rate R1 is provided with a lower limit value (here, 10 fps). In addition, as the size of the region of interest in the blood-flow image corresponding to the lower limit value, the length d of the region of interest in the depth direction (the Y direction in FIG. 2) is provided with an upper limit value (depth index d5). The ROI setting unit 113 stores the lower limit value and the initial value (e.g., 14 fps) of the frame rate R1 provided by the user through the operation unit (not illustrated) before the puncture in its internal memory.

FIG. 4 illustrates an example of the movement amount relation table. In FIG. 4, the movement amount relation table provides four levels of the velocity indices 0 to 3 each corresponding to the value of the movement amount L of the position of the needle tip per unit time. The movement amount relation table also provides four levels of the acceleration indices 0 to 3 each corresponding to the value of the movement velocity V of the position of the needle tip per unit time.

In the movement amount relation table illustrated in FIG. 4, for example, when the acceleration index is zero, if the velocity index increases like 0, 1, 2, to 3, the depth index correspondingly increases like d0, d1, d2, to d3. When the acceleration index increases like 0, 1, 2, to 3, the depth index correspondingly increases like d0, d1, d2, to d3. That is, if the movement amount L (corresponding to the velocity) or the movement velocity V (corresponding to the acceleration) of the position of the needle tip per unit time increases, the depth direction length d, as the size of the region of interest ROI1, gradually increases. Besides, for example, when the velocity index and the acceleration index are 0, 1, 2, and 3, the depth index correspondingly increases like d0, d2, d4 to d5 according to the indices. That is, if both the movement amount L (corresponding to the velocity) and the movement velocity V (corresponding to the acceleration) of the position of the needle tip per unit time increases, the region of interest is changed from ROI1 to ROI1′, and the depth direction length d, as the size of the region of interest, drastically increases (see FIG. 2). When the velocity index is 3 and the acceleration index is 2, the depth index is d5. However, even if the acceleration index is increased from 2 to 3, the depth index is unchanged at d5. In this manner, the depth index is provided with the upper limit value d5. The upper limit of the depth index prevents the frame rate R1 from falling below the lower limit value 10 fps.

When the acceleration index increases, the depth index also increases. Thereby, the size of the region of interest ROI1′ increases in the depth direction, and the blood-flow image thereof is generated. As a result, the operator can view a range including a position largely separated from the position of the needle tip in the depth direction. With this, as illustrated in FIG. 2, when the needle tip TN is rapidly moved to puncture a target TG, even if a blood vessel VN runs ahead of the target TG where the needle tip is being directed, the operator can view the range including the position of the blood vessel VN on the blood-flow image of the region of interest R011′, which has been expanded in the depth direction. Thus, the blood vessel VN is unlikely to be damaged.

Incidentally, the depth index need not necessarily be obtained based on the velocity index and/or the acceleration index with reference to the movement amount relation table. Alternatively, the depth direction length d of the regions of interest ROI1 and ROI1′ in the blood-flow image may be obtained from a function f based on the movement amount L and/or the movement velocity V of the position of the needle tip per unit time. As the function f of the movement amount L and/or the movement velocity V of the position of the needle tip per unit time, the length d is represented by the following expression (2):

d=f(L,V) (2)

In this case also, the length d is provided with an upper limit value. When the length d obtained by above expression (2) exceeds, for example, an upper limit value determined in advance, the ROI setting unit 113 in operation outputs the upper limit value. Thereby, the frame rate R1 is prevented from falling below the lower limit value.

Upon receipt of the region of interest ROI2 in the ultrasound image and the frame rate R2 as well as the regions of interest ROI1 and ROI1′ in the blood-flow image and the frame rate R1, the transmit/receive controller 114 controls the transmitter 103 and the receiver 102 to perform B-mode imaging and Doppler mode imaging in time division. For example, the transmit/receive controller 114 controls the transmitter 103 and the receiver 102 to perform B-mode imaging once each time Doppler mode imaging is performed a predetermined number of times (e.g., four times).

### (B-Mode Image Processor 104, Doppler Image Processor 106)

The B-mode image processor 104 generates a three-dimensional ultrasound image based on a beam signal acquired by B-mode imaging, and stores it in the B-mode image storage 105. The Doppler image processor 106 generates a three-dimensional blood-flow image based on a beam signal acquired by Doppler mode imaging, and stores it in the Doppler image storage 107.

### (Puncture Guideline Generator 115)

Upon receipt of a direction DR in which the needle tip NT has been moved and the movement amount of the position of the needle tip NT obtained by the movement amount calculator 112, the puncture guideline generator 115 generates a puncture guideline in the three-dimensional ultrasound image, and stores it in the puncture guideline storage 116.

### (Cross-Section Position Setting Unit 117, Cross-Section Image Processor 108)

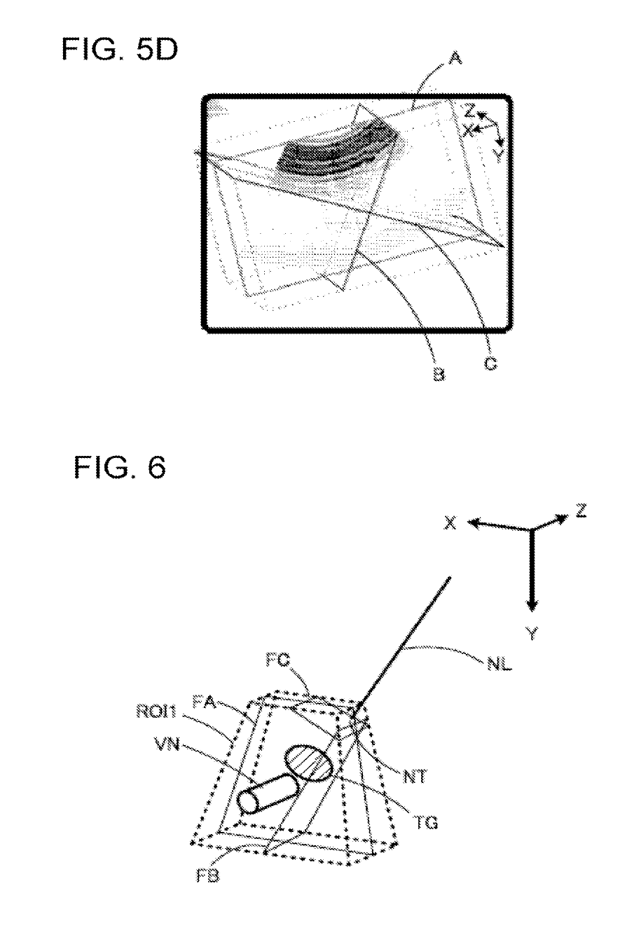

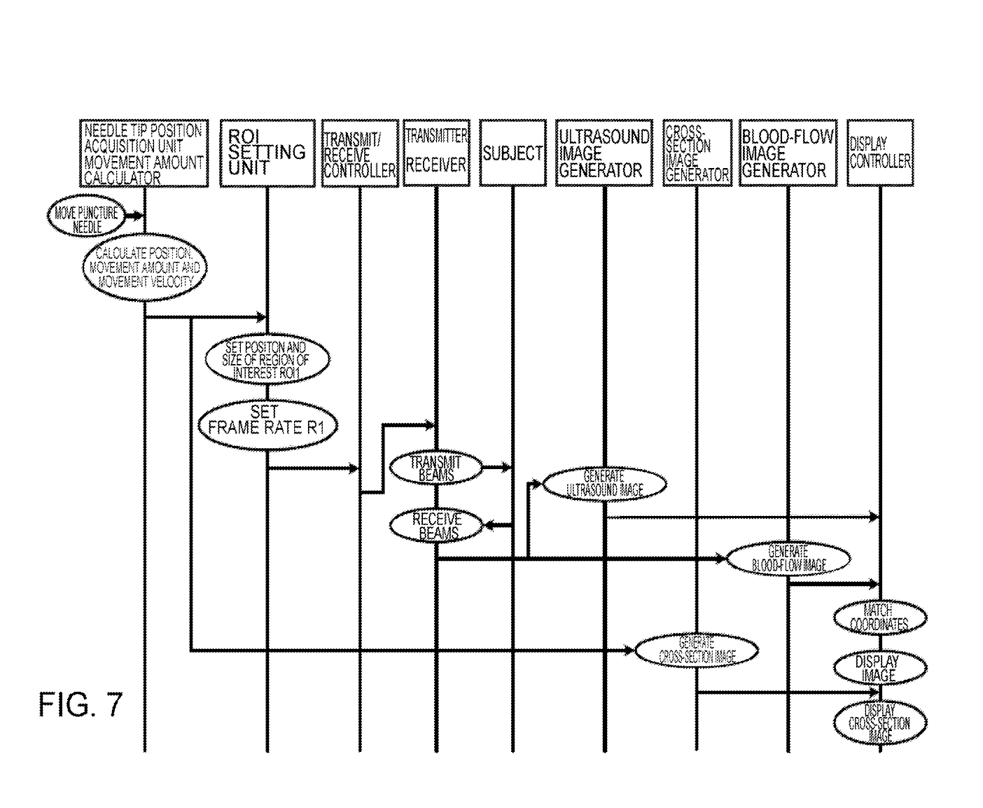

FIG. 5A illustrates an example of a three-dimensional ultrasound image of a cross-section A. FIG. 5B illustrates an example of a three-dimensional ultrasound image of a cross-section B. FIG. 5C illustrates an example of a three-dimensional ultrasound image of a cross-section C. FIG. 5D is a schematic diagram illustrating a positional relationship among the cross-sections A, B and C. FIG. 6 is a schematic diagram illustrating a positional relationship among the ultrasound image, and the cross-sections A, B and C. The cross-section C is a cross-section perpendicular to the direction DR in which the puncture needle NL has been moved (also perpendicular to a puncture guideline GL). The cross-section A is perpendicular to the cross-section C and extending along the width direction (X direction). The cross-section B is perpendicular to the cross-sections A and C. FIG. 5A illustrates the regions of interest ROI1 and ROI1′ corresponding to the position of the needle tip. FIG. 6 illustrates the cross-section C as a cross-section that is perpendicular to the axis of the puncture needle NL and located at a predetermined distance from the position of the tip of the puncture needle NL.

Upon receipt of the position of the needle tip NT on the three-dimensional coordinates obtained per unit time and the direction in which the needle tip NT has been moved calculated by the movement amount calculator 112, the cross-section position setting unit 117 sets the position of a cross-section (cross-section C) an image of which is to be displayed, the angle (corresponding to the direction in which needle tip has been moved), the number of cross-sections (here, three cross-sections A, B and C), and the position of each cross-section. As the distance between the position of the needle tip and the position of the cross-section C, for example, any distance from 0 mm to 20 mm is set in the cross-section position setting unit 117 by the user through the operation unit (not illustrated). Upon receipt of the position of each cross-section and the like thus set, the cross-section image processor 108 generates cross-section images of the cross-sections A, B and C, and stores them in the cross-section image storage 109.

### (Display 110)

The display 110 includes a display controller. The display controller superimposes the three-dimensional blood-flow image on the three-dimensional ultrasound image to be displayed on the monitor (not illustrated) together with the puncture guideline GL of the puncture needle NL (see FIG. 2). Further, the display 110 displays the cross-section images of the cross-sections A, B and C on the monitor (see FIGS. 5D and 6).

### (Operation)

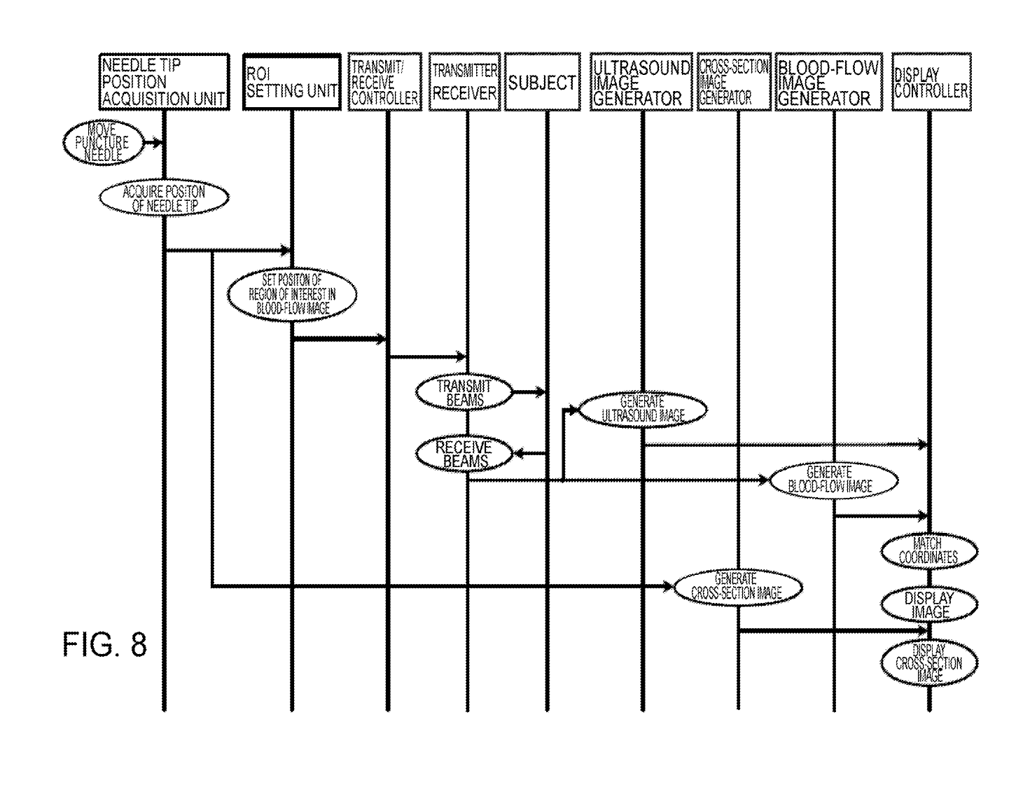

Next, with reference to FIGS. 5A to 5D and 7, a description is given of the operation of each unit of the ultrasound diagnosis apparatus of the first embodiment.

At the start of the puncture, the needle tip position acquisition unit 111 acquires the position of the needle tip NT on the coordinates of a three-dimensional ultrasound image. The ROI setting unit 113 sets the region of interest ROI2 (fixed) in the ultrasound image, the frame rate R2 (fixed), the depth index (initial value: d3) as the size of the regions of interest ROI1 and ROI1′ in the blood-flow image, and the frame rate R1 (initial value: 14 fps). The transmit/receive controller 114 controls the transmitter 103 and the receiver 102 based on conditions including them. The B-mode image processor 104 generates a three-dimensional ultrasound image based on a reception beam acquired. The Doppler image processor 106 generates a three-dimensional blood-flow image based on the reception beam. The display controller (not illustrated) superimposes the three-dimensional blood-flow image on the three-dimensional ultrasound image to be displayed on the monitor. It is not to be mentioned here that the puncture guideline GL of the puncture needle NL is generated based on the movement amount L of the position of the needle tip and the like and is displayed.

FIG. 7 is a timing chart illustrating the operation of each unit from the movement of the puncture needle NL until the display of a cross-section image. In the puncture, a three-dimensional ultrasound image is generated based on the size (fixed) of the region of interest ROI2 in the ultrasound image and the frame rate R2 (fixed), and is displayed on the monitor. At the start of the puncture, a three-dimensional blood-flow image is generated based on the size (initial value) of the region of interest ROI1 in the blood-flow image and the frame rate R1 (initial value), and is displayed on the monitor.

In the following, the generation of the three-dimensional blood-flow image displayed as being superimposed on the three-dimensional ultrasound image is mainly described.

### (Calculation of Position, Movement Amount, Etc.)

As illustrated in FIG. 7, when the puncture needle NL is moved in the puncture, the needle tip position acquisition unit 111 acquires the position of the needle tip NT on the three-dimensional coordinates per unit time base on detection results obtained by the movement amount sensor and the passing sensor. The movement amount calculator 112 calculates the movement amount L and the movement velocity V per unit time based on the position of the needle tip NT on the three-dimensional coordinates obtained per unit time.

### (Setting of Position and Size of Regions of Interest ROI1 and ROI1′)

The ROI setting unit 113 obtains the position of the region of interest in the blood-flow image and the depth index from the velocity index and the acceleration index based on the position of the needle tip NT on the three-dimensional coordinates and the movement amount L and/or the movement velocity V of the position of the needle tip per unit time with reference to the movement amount relation table as illustrated in FIG. 4. With reference to the rate relation table as illustrated in FIG. 3, the ROI setting unit 113 obtains the frame rate R1 corresponding to the depth index. The ROI setting unit 113 outputs the position and the size of the regions of interest ROI1 and ROI1′ as well as the frame rate R1 to the transmit/receive controller 114.

Thereby, the size (the length d in the depth direction) of the regions of interest R011 and R011′ in the blood-flow image becomes a length corresponding to the movement amount L and/or the movement velocity V of the position of the needle tip per unit time obtained by the ROI setting unit 113.

As a result of the calculation by the movement amount calculator 112, for example, when the velocity index is 2 and the acceleration index is 1, the ROI setting unit 113 obtains the depth index d3 corresponding to the velocity index and the acceleration index with reference to the movement amount relation table. In addition, the ROI setting unit 113 obtains 14 fps as the frame rate R1 corresponding to the depth index d3 with reference to the rate relation table.

### (Beam Transmission, Beam Reception)

Upon receipt of the position and the size of the region of interest ROI1 in the blood-flow image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (Doppler mode imaging). Besides, upon receipt of the size of the region of interest ROI2 in the ultrasound image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (B-mode imaging). As described above, B-mode imaging and Doppler mode imaging are performed in time division.

### (Generation of Ultrasound Image, Blood-Flow Image, Coordinate Matching, Etc.)

The B-mode image processor 104 generates a three-dimensional ultrasound image based on a beam signal acquired by B-mode imaging. The Doppler image processor 106 generates a three-dimensional blood-flow image based on a beam signal acquired by Doppler mode imaging. The display controller (not illustrated) matches the coordinates of the three-dimensional ultrasound image and the three-dimensional blood-flow image, and displays the blood-flow image of the region of interest ROI1 corresponding to the position of the needle tip superimposed on the ultrasound image. FIG. 5A illustrates the blood-flow image of the region of interest ROI1 together with a marker M that indicates the position of the needle tip located at the upper edge of the region of interest ROI1.

As a result of the calculation by the movement amount calculator 112, for example, when the velocity index is 2 and the acceleration index is 3, the ROI setting unit 113 obtains the depth index d5 corresponding to the velocity index and the acceleration index with reference to the movement amount relation table. In addition, the ROI setting unit 113 obtains 10 fps as the frame rate R1 corresponding to the depth index d5 with reference to the rate relation table.

Upon receipt of the position and the size of the region of interest ROI1′ in the blood-flow image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (Doppler mode imaging). Besides, upon receipt of the size of the region of interest ROI2 in the ultrasound image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (B-mode imaging). As described above, B-mode imaging and Doppler mode imaging are performed in time division.

The B-mode image processor 104 generates a three-dimensional ultrasound image based on a beam signal acquired by B-mode imaging. The Doppler image processor 106 generates a three-dimensional blood-flow image based on a beam signal acquired by Doppler mode imaging. The display controller (not illustrated) matches the coordinates of the three-dimensional ultrasound image and the three-dimensional blood-flow image, and displays the blood-flow image of the region of interest ROI1′ corresponding to the position of the needle tip superimposed on the ultrasound image. FIG. 2 illustrates the regions of interest R011 and ROI1′ in the blood-flow image having different lengths d according to the velocity index and the acceleration index. In FIG. 2, the marker M (see FIG. 5A), which indicates the position of the needle tip and is located at the upper edge of the region of interest ROI1′, is not illustrated. The region of interest ROI1 is indicated by a dashed line in the region of interest ROI1′ to compare their sizes.

### (Generation of Tomographic Image)

Upon receipt of the position of the needle tip, the cross-section image processor 108 generates cross-section images of the cross-sections A, B and C. The display controller (not illustrated) displays the cross-section images on the monitor (see FIGS. 5A to 5C). Further, as illustrated in FIG. 5A, the cross-section image processor 108 obtains the position PC of the cross-section C on the cross-section A. The display controller displays the position PC of the cross-section C superimposed on the cross-section image of the cross-section A.

In this manner, the cross-section image (particularly, the cross-section image of the cross-section C) is displayed. This allows the operator to view a target (in some cases, blood vessel) at a predetermined distance (i.e., at the position of the cross-section C) from the position of the needle tip in the depth direction. Further, the position of the cross-section C is displayed on another cross-section (cross-section A or B). Thereby, the distance between the needle tip and the cross-section C can be estimated from the position of the cross-section C displayed on another cross-section

The cross-section image processor 108 may generate, on a surface perpendicular to the direction DR in which the puncture needle NL has been moved (also perpendicular to the puncture guideline GL) in a position at a predetermined distance from the position of the needle tip in the depth direction (Y direction), a projection image of a blood-flow image located in the depth direction from the surface. The display controller may display the projection image. With this, the operator can view one or more targets (blood vessel VN illustrated in FIG. 5A) located around the position of the needle tip NT and also in the depth direction therefrom.

### Second Embodiment

With reference to the drawings, a description is given of an ultrasound diagnosis apparatus according to a second embodiment. In the second embodiment, differences from the ultrasound diagnosis apparatus of the first embodiment are mainly described, and the same description may not be repeated.

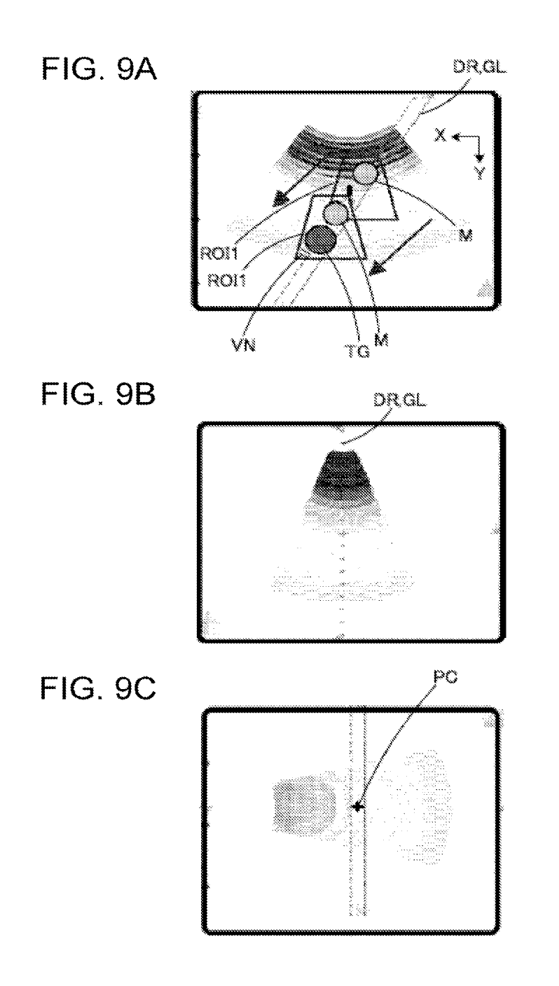

FIG. 8 is a timing chart illustrating the operation of each unit of the ultrasound diagnosis apparatus of the second embodiment from the movement of the puncture needle NL until the display of a cross-section image.

In the first embodiment, the needle tip position acquisition unit 111 acquires the position of the needle tip on the coordinates per unit time. The movement amount calculator 112 calculates the movement amount L and the movement velocity V of the needle tip per unit time based on the position of the needle tip. During the puncture, each time the needle tip position acquisition unit 111 acquires the position of the needle tip, the ROI setting unit 113 sets the region of interest ROI1 of the blood-flow image, which has the length d in the depth direction according to the movement amount L and/or the movement velocity V of the position of the needle tip per unit time, in a position corresponding to the position of the needle tip such that the needle tip is located in the upper edge of the region of interest ROI1 (the head portion of the region of interest in the blood-flow image having a substantially truncated pyramid shape).

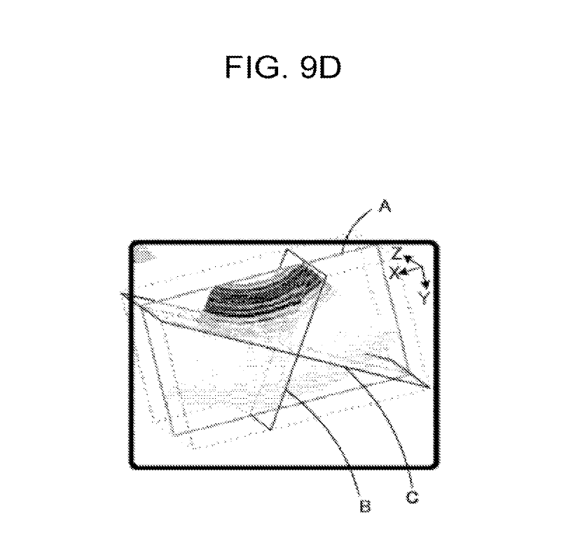

On the other hand, in the second embodiment, the ultrasound diagnosis apparatus is not provided with the movement amount calculator 112 as illustrated in FIG. 8. During the puncture, each time the needle tip position acquisition unit 111 acquires the position of the needle tip, the ROI setting unit 113 obtains the region of interest ROI1 in the blood-flow image, which includes the position of the needle tip and extends along the movement direction of the needle tip (the puncture guideline). The ROI setting unit 113 sends the region of interest ROI1 in the blood-flow image according to the position of the needle tip to the transmit/receive controller 114. Thereby, the region of interest ROI1 is set to include the position of the needle tip. That is, the needle tip is located at the upper edge of the region of interest ROI1 (the head portion of the region of interest in the blood-flow image having a substantially truncated pyramid shape) as illustrated in FIG. 9A. In the second embodiment, the ROI setting unit 113 changes the position of the region of interest ROI1 in the blood-flow image according to the position of the needle tip. However, the size (lengths d, w, u) of the region of interest ROI1 is constant, and do not vary depending on the movement amount L and/or the movement velocity V of the position of the needle tip per unit time.

FIG. 9A is an example of a three-dimensional ultrasound image of the cross-section A. As illustrated in FIG. 9A, the display controller (not illustrated) displays the region of interest ROI1 in the blood-flow image together with the marker M for identifying the position of the needle tip. The marker M is displayed at the upper edge of the region of interest R011.

As the region of interest ROI1 in the blood-flow image moves to a position corresponding to the position of the needle tip, the display controller displays a target located in the depth direction from the position of the needle tip. Thus, the operator can check whether the target includes the blood vessel VN (see FIG. 9A).

FIG. 9A illustrates an example of a three-dimensional ultrasound image of the cross-section A. FIG. 9B illustrates an example of a three-dimensional ultrasound image of the cross-section B. FIG. 9C illustrates an example of a three-dimensional ultrasound image of the cross-section C. FIG. 9D is a schematic diagram illustrating a positional relationship among the ultrasound image, and the cross-sections A, B and C.

As illustrated in FIGS. 9A to 9D, also in the second embodiment, the cross-section image processor 108 generates cross-section images of the cross-sections A, B and C. The display 110 also displays the cross-section images of the cross-sections A, B and C on the monitor.

In the embodiment described above, a blood-flow image is displayed as being superimposed on an ultrasound image, the blood-flow image may be displayed as being superimposed on a morphology image (CT image or MRI image). In this case, the marker M for identifying the position of the needle tip is displayed together with the blood-flow image. Thereby, a positional relationship between a blood vessel and the needle tip is displayed on the morphology image in real time. Thus, it is possible to figure out whether a blood vessel runs ahead of the direction in which the needle chip is being directed.

Besides, in the above embodiment, an example is described in which the display controller (not illustrated) displays a three-dimensional blood-flow image superimposed on a three-dimensional ultrasound image; however, this is not so limited. For example, the display controller may display a two-dimensional or a three-dimensional blood-flow image superimposed on a three-dimensional ultrasound image. With this, even if the puncture needle NL is deviated from the two-dimensional ultrasound image, the operator can visually check whether there is a blood vessel around the needle tip in the two- or three-dimensional blood-flow image. In addition, the display controller may display a two-dimensional blood-flow image superimposed on a three-dimensional ultrasound image. As a result, for example, if the direction DR in which the puncture needle NL has been moved is parallel to a two-dimensional XY plane, the operator can visually check whether there is a blood vessel in the direction in which the needle tip is being directed with the two-dimensional blood-flow image. Moreover, the frame rate of the two-dimensional blood-flow image can be kept high as compared to a three-dimensional blood-flow image. Thus, the blood-flow image is displayed in real time.

Further, in the above embodiment, for example, the distance between the position of the needle tip and the position of the cross-section C is determined to be any distance from 0 mm to 20 mm. The cross-section image of the cross-sections C in a predetermined position is displayed. However, this is not so limited. The cross-section position setting unit 117 may determine the distance between the position of the needle tip and the position of the cross-section C in stages like 0 mm, 5 mm, 10 mm, 15 mm, and 20 mm. In this case, the cross-section image processor 108 generates a cross-section image of the cross-section C at each of the distances determined in stages. The display controller selectively displays, on the monitor, one or more cross-section images of the cross-section C at the distances. With this, by entering a desired distance to the cross-section position setting unit 117 according to the movement amount of the needle tip NT moved in the depth direction, the operator can select the cross-section image of the cross-section C in a position suitable for him/her. In addition, when two or more cross-section images are selected to be displayed and, for example, a blood vessel image is not displayed in the cross-section image of the cross-section C at a distance of 5 mm, but in the cross-section image of the cross-section C at a distance of 15 mm, the operator can recognize that the needle tip NT can be moved 10 mm in the depth direction.

### Third Embodiment

A description is given of an ultrasound diagnosis apparatus according to a third embodiment. In the third embodiment, differences from the ultrasound diagnosis apparatus of the first embodiment are mainly described, and the same description may not be repeated.

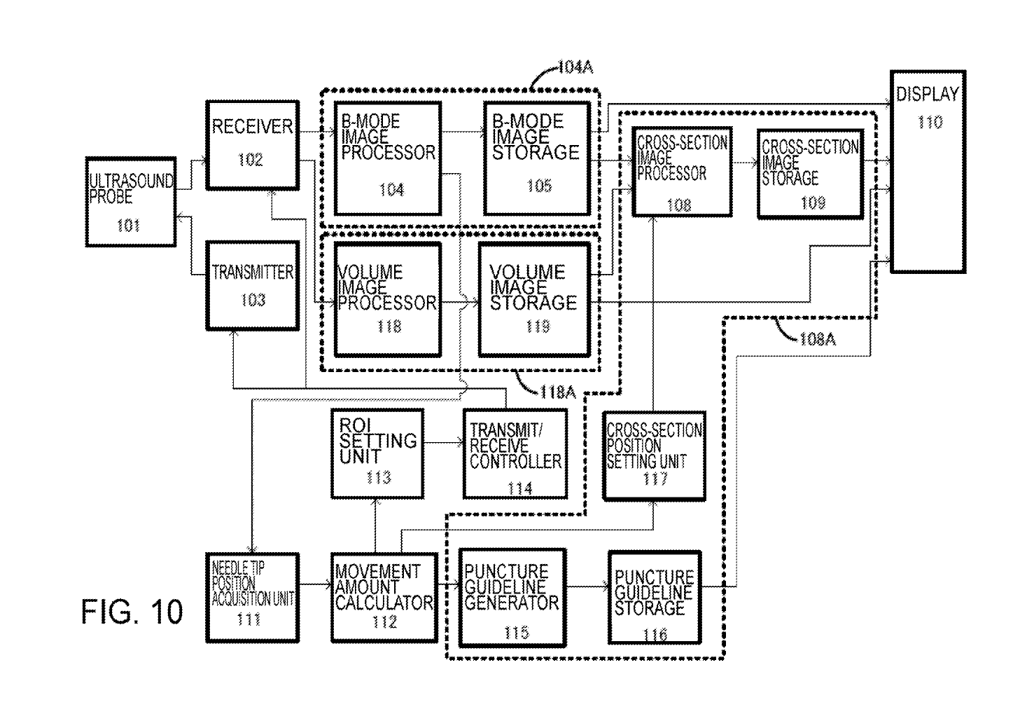

FIG. 10 is a block diagram illustrating the configuration of the ultrasound diagnosis apparatus of the third embodiment. The ultrasound diagnosis apparatus of the third embodiment includes a volume image generator 118A in place of the blood-flow image generator 106A of the first embodiment. The volume image generator 118A includes a volume image processor 118 and a volume image storage 119.

The transmit/receive controller 114 controls the transmitter 103 and the receiver 102 to perform B-mode imaging and volume mode imaging in time division. For example, B-mode imaging is performed once each time volume mode imaging is performed a predetermined number of times (e.g., four times) under the control of the transmit/receive controller 114.

In the volume mode imaging, the transmit/receive controller 114 receives a region of interest in a volume image and a frame rate, and obtains transmission conditions (delay time, the number of transmissions, etc.) and reception conditions (reception channel, delay amount for each channel, etc.). The transmit/receive controller 114 controls the transmitter 103 and the receiver 102 based on these conditions to transmit ultrasound beams and receive reflected waves a plurality of times (e.g., four times) per one scan line to thereby repeat scan for one still image having a required number of scan lines to render a three-dimensional form of the region of interest at the frame rate of the volume image. The volume image corresponds to an example of ROI image in the claims.

The region of interest in the volume image of the third embodiment corresponds to the regions of interest ROI1 and ROI1′ in the blood-flow image illustrated in FIG. 2. The frame rate of the volume image corresponds to the frame rate R1 of the first embodiment.

Upon receipt of the region of interest ROI2 in an ultrasound image and the frame rate R2 as well as the regions of interest ROI1 and ROI1′ in a blood-flow image and the frame rate R1, the transmit/receive controller 114 controls the transmitter 103 and the receiver 102 to perform B-mode imaging and volume mode imaging in time division. For example, the transmit/receive controller 114 controls the transmitter 103 and the receiver 102 to perform B-mode imaging once each time volume mode imaging is performed a predetermined number of times (e.g., four times).

The volume image processor 118 generates a volume image based on a beam signal acquired by volume mode imaging, and stores it in the volume image storage 119.

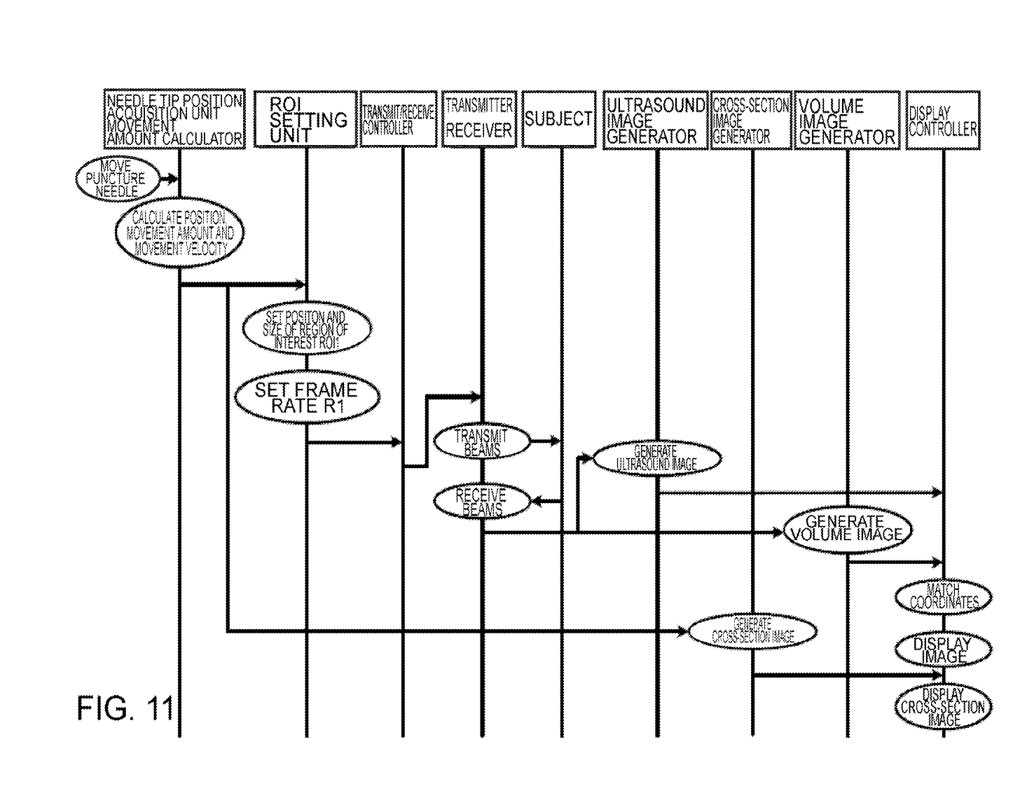

Next, with reference to FIG. 11, a description is given of the operation of each unit of the ultrasound diagnosis apparatus of the third embodiment.

At the start of the puncture, the needle tip position acquisition unit 111 acquires the position of the needle tip NT on the coordinates of a three-dimensional ultrasound image. The ROI setting unit 113 sets the region of interest ROI2 (fixed) in the ultrasound image, the frame rate R2 (fixed), the depth index (initial value: d3) as the size of the regions of interest ROI1 and ROI1′ in the blood-flow image, and the frame rate R1 (initial value: 14 fps). The transmit/receive controller 114 controls the transmitter 103 and the receiver 102 based on conditions including them. The B-mode image processor 104 generates a three-dimensional ultrasound image based on a reception beam acquired. The volume image processor 118 generates a volume image based on the reception beam.

FIG. 11 is a timing chart illustrating the operation of each unit from the movement of the puncture needle NL until the display of a cross-section image. In the puncture, a three-dimensional ultrasound image is generated based on the size (fixed) of the region of interest ROI2 in the ultrasound image and the frame rate R2 (fixed), and is displayed on the monitor. At the start of the puncture, a volume image is generated based on the size (initial value) of the region of interest ROI1 in the volume image and the frame rate R1 (initial value), and is displayed on the monitor.

The ROI setting unit 113 obtains the position of the region of interest in the volume image and the depth index from the velocity index and the acceleration index based on the position of the needle tip NT on the three-dimensional coordinates and the movement amount L and/or the movement velocity V of the position of the needle tip obtained per unit time with reference to the movement amount relation table as illustrated in FIG. 4. With reference to the rate relation table as illustrated in FIG. 3, the ROI setting unit 113 obtains the frame rate R1 corresponding to the depth index. The ROI setting unit 113 outputs the position and the size of the regions of interest ROI1 and ROI1′ as well as the frame rate R1 to the transmit/receive controller 114.

Thereby, the size (the length d in the depth direction) of the regions of interest ROI1 and ROI1′ in the volume image becomes a length corresponding to the movement amount L and/or the movement velocity V of the position of the needle tip obtained per unit time by the ROI setting unit 113.

Upon receipt of the position and the size of the region of interest ROI1 in the blood-flow image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (volume mode imaging). Besides, upon receipt of the size of the region of interest ROI2 in the ultrasound image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (B-mode imaging). As described above, B-mode imaging and volume mode imaging are performed in time division.

The B-mode image processor 104 generates a three-dimensional ultrasound image based on a beam signal acquired by B-mode imaging. The volume image processor 118 generates a volume image based on a beam signal acquired by volume mode imaging.

Upon receipt of the position and the size of the region of interest ROI1′ in the blood-flow image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (volume mode imaging). Besides, upon receipt of the size of the region of interest ROI2 in the ultrasound image and the frame rate R1, the transmit/receive controller 114 obtains transmission/reception conditions. The transmit/receive controller 114 controls the transmitter 103 and the receiver 102 based on the conditions to transmit ultrasound beams and receive reflected waves (B-mode imaging). As described above, B-mode imaging and volume mode imaging are performed in time division.

The B-mode image processor 104 generates a three-dimensional ultrasound image based on a beam signal acquired by B-mode imaging. The volume image processor 118 generates a volume image based on a beam signal acquired by volume mode imaging. The display controller (not illustrated) matches the coordinates of the three-dimensional ultrasound image and the volume image, and displays the volume image of the region of interest ROI1′ corresponding to the position of the needle tip superimposed on the ultrasound image.

According to at least one of the above embodiments, in puncture under ultrasound echo-guidance, a blood-flow image or a volume image can be displayed in real time while the frame rate is kept high. This removes factors that prolong a surgery, thus imposing no burden on the operator.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus, comprising processing circuitry configured to:
transmit ultrasound beams to a subject into which a puncture needle is inserted while scanning the subject;
receive signals reflected from the subject;
successively acquire a position of a needle tip of the puncture needle;
set a region of interest at least in a direction in which the puncture needle is inserted;
generate a region of interest (ROI) image of the region of interest according to the position of the needle tip based on the signals; and
display the ROI image.

2. The ultrasound diagnosis apparatus of claim 1, wherein the processing circuitry is further configured to generate a blood-flow image that represents blood-flow information of the region of interest as the ROI image.

3. The ultrasound diagnosis apparatus of claim 1, wherein the processing circuitry is further configured to generate a volume image of the region of interest as the ROI image.

4. The ultrasound diagnosis apparatus of claim 1, wherein the processing circuitry is further configured to:
generate the ROI image at a rate equal to or above a predetermined lower limit value, the rate corresponding to the number of images generated per unit time; and
set a size of the region of interest such that the rate does not fall below the lower limit value.

5. The ultrasound diagnosis apparatus of claim 1, wherein the processing circuitry is further configured to set a size of the region of interest according to movement amount and/or movement velocity of the position of the needle tip per unit time.

6. The ultrasound diagnosis apparatus of claim 5, wherein the processing circuitry is further configured to set a length in depth direction form the position of the needle tip as the size of the region of interest.

7. The ultrasound diagnosis apparatus of claim 1, wherein the processing circuitry is further configured to:
scan the subject in width direction perpendicular to depth direction while transmitting ultrasound beams in the depth direction;
further receive signals reflected from the subject;
generate an ultrasound image based on the signals; and
display the ROI image of the region of interest corresponding to the position of the needle tip in the ultrasound image, the ROI image being superimposed on the ultrasound image.

8. The ultrasound diagnosis apparatus of claim 1, wherein the processing circuitry is further configured to:
obtain a direction in which the puncture needle has moved based on the position of the needle tip;
generate a cross-section image of a surface perpendicular to the direction in a position at a predetermined distance from the position of the needle tip in the direction based on the ROI image of the region of interest corresponding to the position of the needle tip; and
display the cross-section image.

9. The ultrasound diagnosis apparatus of claim 8, wherein the processing circuitry is further configured to:
generate an image of a surface, which is perpendicular to the cross-section image and along width direction perpendicular to depth direction, based on the ROI image of the region of interest corresponding to the position of the needle tip;
obtain a position of the cross-section image in the image;
display the image, which is perpendicular to the cross-section image, with the position of the cross-section image.

10. The ultrasound diagnosis apparatus of claim 1, wherein the processing circuitry is further configured to:
obtain a direction in which the puncture needle has moved based on the position of the needle tip;
generate, on a surface perpendicular to the direction in a position at a predetermined distance from the position of the needle tip in the direction, a projection image of a blood-flow image located in depth direction from the position at the predetermined distance based on the ROI image of the region of interest corresponding to the position of the needle tip; and
display the projection image.
